(19) United States
(12) Patent Application Publication
(10) Pub. No.: US 2019/0200875 A1
(43) Pub. Date: Jul. 4, 2019

(54) PHOTOACOUSTIC APPARATUS, INFORMATION PROCESSING APPARATUS, AND METHOD

(71) Applicant: CANON KABUSHIKI KAISHA, Tokyo (JP)

(72) Inventors: Kohtaro Umezawa, Tokyo (JP); Norihiko Utsunomiya, Machida-shi (JP); Yohei Hashizume, Tokyo (JP); Fumitaro Masaki, Brookline, MA (US); Toshifumi Fukui, Yokohama-shi (JP)

(21) Appl. No.: 16/220,409

(22) Filed: Dec. 14, 2018

(30) Foreign Application Priority Data
Dec. 28, 2017 (JP) 2017-253591

(51) Int. Cl. A61B 5/00; A61B 8/00; A61B 8/08

(52) U.S. Cl. CPC A61B 5/0095; A61B 5/0059; A61B 8/483; A61B 8/429; A61B 8/4281

(57) ABSTRACT

Disclosed is a photoacoustic apparatus including: a photoacoustic probe having a light irradiating unit that irradiates light onto an object and an acoustic wave receiving unit that receives an acoustic wave generated from the object by the irradiation of the light; a specific information acquiring unit that acquires specific information of the object based on the acoustic wave; an order acquiring unit that acquires an order relating to the acquisition of the specific information from an ordering system; an object information acquiring unit that acquires object information relating to the object from an electronic medical record; and a control condition determining unit that determines a control condition of photoacoustic probe based on the order and the object information.

### BACKGROUND OF THE INVENTION

### Field of the Invention

The present invention relates to a photoacoustic apparatus, an information processing apparatus, and a method.

### Description of the Related Art

In a medical field, the apparatuses of various modalities have been developed to image objects and generate images useful for diagnoses. Measurement results by the apparatuses could change according to the states of objects. Therefore, operators using the apparatuses set parameters according to the states of the objects to adjust the control conditions of the apparatuses. For example, in some Mill apparatuses, an available mode for image capturing differs depending on the type of a cardiac pacemaker used by an object. In addition, in X-ray image capturing apparatuses, an X-ray tube voltage, a current value, an image capturing time, or the like is required to be set, as appropriate, according to an imaging target or a body thickness. An X-ray diagnostic apparatus described in Japanese Patent Application Laid-open No. 2000-308631 sets control conditions for imaging based on an examination order acquired by communicating with an ordering system in a hospital.

In recent years, research on a technology called photoacoustic imaging for imaging a constituent component inside a living body, has been advanced as a new modality. A photoacoustic apparatus using the photoacoustic imaging irradiates light onto an object, receives photoacoustic waves generated when irradiation light is propagated and diffused inside the object and absorbed in a light absorber (such as blood vessels or a skin), and converts the received photoacoustic waves into an electrical signal. By analyzing the electrical signal, it is possible to generate image data reflecting an optical characteristic distribution inside the object and visualize a constituent component.

Patent Literature 1: Japanese Patent Application Laid-open No. 2000-308631

### SUMMARY OF THE INVENTION

In photoacoustic measurement as well, there is a case that a measurement result or a generated image changes according to object information showing the state of an object. For example, when an object contains a large amount of a component (such as melanin and a contrast agent administered for measurement by other modalities) that easily absorbs light, the intensity of a hemoglobin component in blood that is to be observed by a doctor reduces. In addition, there is a case that the control conditions of a photoacoustic apparatus are required to be changed according to the characteristics or symptom of an object. For example, when an object has photosensitivity, the amount of irradiation light is required to be reduced.

However, object information showing the state of an object is not necessarily contained in an imaging order set in an ordering system. That is, there is a case that object information exists only in an electronic medical record or the head of a doctor. In addition, there is a case that a plurality of object information having an influence on light absorption or image formation exist or a plurality of control conditions required to be changed corresponding to certain object information exist. Therefore, it is difficult for an operator (engineer) to set control conditions in consideration of all elements.

The present invention has been made in view of the above problems. It is an object of the present invention to provide a technology for easily performing measurement complying with an imaging order in a photoacoustic apparatus.

The present invention provides a photoacoustic apparatus comprising:

a photoacoustic probe configured to include a light irradiating unit that irradiates light onto an object and an acoustic wave receiving unit that receives an acoustic wave generated from the object by the irradiation of the light;

a specific information acquiring unit configured to acquire specific information of the object based on the acoustic wave;

an order acquiring unit configured to acquire an order relating to the acquisition of the specific information from an ordering system;

an object information acquiring unit configured to acquire object information relating to the object from an electronic medical record; and

a control condition determining unit configured to determine a control condition of photoacoustic probe based on the order and the object information.

The present invention also provides an information processing apparatus comprising:

a control condition determining unit configured to acquire, from an ordering system, an order relating to acquisition of specific information of an object generated based on a photoacoustic wave generated when light is irradiated onto the object, acquire object information relating to the object from an electronic medical record, and determine a control condition of a photoacoustic probe to acquire the photoacoustic wave based on the order and the object information.

The present invention also provides an information processing method comprising:

acquiring, from an ordering system, an order relating to acquisition of specific information of an object generated based on a photoacoustic wave generated when light is irradiated onto the object;

acquiring object information relating to the object from an electronic medical record; and

determining a control condition of a photoacoustic probe to acquire the photoacoustic wave based on the order and the object information.

According to an embodiment of the present invention, it is possible to provide a technology for easily performing measurement complying with an imaging order in a photoacoustic apparatus.

Further features of the present invention will become apparent from the following description of exemplary embodiments with reference to the attached drawings.

### BRIEF DESCRIPTION OF THE DRAWINGS

FIG. 1 is a block diagram showing the schematic configuration of a photoacoustic apparatus;

FIG. 2 is a flowchart showing the outline of processing;

FIGS. 3A and 3B are block diagrams showing the internal configurations of an information processing unit and a signal processing unit;

FIG. 4 is a diagram showing the absorption spectrums and the contrasts of two components; and

FIG. 5 is a diagram for describing the absorption spectrum of ICG.

### DESCRIPTION OF THE EMBODIMENTS

Hereinafter, preferred embodiments of the present invention will be described with reference to the drawings. However, the sizes, materials, shapes, their relative arrangements, or the like of constituents described below may be appropriately changed according to the configurations or various conditions of apparatuses to which the present invention is applied. Accordingly, the sizes, materials, shapes, their relative arrangements, or the like do not intend to limit the scope of the present invention to the following description.

The present invention relates to a technology for receiving photoacoustic waves propagated from an object with a photoacoustic probe, storing a photoacoustic signal that is a signal based on the photoacoustic waves, and generating specific information inside the object from the photoacoustic signal. The present invention is also grasped as an information processing apparatus for controlling a photoacoustic apparatus. The present invention is also grasped as a method for generating or displaying an image showing specific information inside an object. The present invention is also grasped as a photoacoustic probe, a photoacoustic apparatus, or a method for controlling the same. The present invention is also grasped as a method for acquiring a photoacoustic signal or an information processing method associated with the control conditions of a photoacoustic apparatus. The present invention is also grasped as a program that causes an information processing apparatus including hardware resources such as a CPU and a memory to perform these methods or a non-transitory computer readable storage medium that stores the program.

The photoacoustic apparatus according to the present invention includes a photoacoustic apparatus that makes use of a photoacoustic effect in which photoacoustic waves generated inside an object by the irradiation of light (electromagnetic waves) onto the object are received and the specific information of the object is acquired as image data. Specific information in the photoacoustic apparatus is the information of a specific value corresponding to each of a plurality of positions inside an object, the information being generated using a signal derived from received photoacoustic waves. A photoacoustic signal according to the present invention is a concept including photoacoustic waves generated by the irradiation of light, a photoacoustic signal converted from the photoacoustic waves, and any signal acquired by subjecting the photoacoustic signal to various signal processing such as addition and correction.

Image data acquired from a photoacoustic signal is image data showing the spatial distribution of at least one object information such as the generated sound pressure (initial sound pressure), absorption energy density, and absorption coefficient of photoacoustic waves. Note that photoacoustic image data showing spectral information such as the concentration of a substance constituting an object is acquired based on photoacoustic waves generated by the irradiation of light having a plurality of different wavelengths. The photoacoustic image data showing the spectral information may be oxygen saturation, a value obtained by weighting the oxygen saturation with intensity such as an absorption coefficient, total hemoglobin concentration, oxyhemoglobin concentration, or deoxyhemoglobin concentration. The photoacoustic image data showing the spectral information may also be glucose concentration, collagen concentration, melanin concentration, or the volume fraction of fat or water. More specifically, the melanin concentration may be separately acquired as eumelanin concentration and pheomelanin concentration.

Based on the spectral information of respective positions inside an object, a two-dimensional or three-dimensional specific information distribution is acquired. Distribution data can be generated as image data. The specific information may be calculated as the distribution information of respective positions inside an object rather than being calculated as numerical data. That is, the specific information is distribution information such as an initial sound pressure distribution, an energy absorption density distribution, an absorption coefficient distribution, and an oxygen saturation distribution. The distribution data is also called photoacoustic image data or reconfiguration image data.

In the present invention, acoustic waves are typically ultrasonic waves and include elastic waves called sound waves or acoustic waves. An electrical signal converted from the acoustic waves by a transducer or the like is also called an acoustic signal. However, in the specification, the ultrasonic waves or acoustic waves do not intend to limit the wavelengths of such elastic waves. The acoustic waves generated by the photoacoustic effect are called photoacoustic waves or light ultrasonic waves. An electrical signal derived from the photoacoustic waves is also called a photoacoustic signal. In the specification, the “photoacoustic signal” may include data acquired by subjecting a signal converted from the photoacoustic waves to various processing.

### First Embodiment

The photoacoustic apparatus according to the present embodiment determines control conditions so as to comply with an imaging order from a doctor based on object information showing the state of an object.

In the following description, the “imaging order” is an image acquisition instruction using an ordering system by a doctor. The imaging order may be a part of an examination order in a medical examination or a through physical examination. The imaging order includes an object, a modality, a measurement target, or the like. In addition, the imaging order may include further detailed instructions such as image precision.

As a measurement target, a doctor may designate concrete specific information (such as oxygen saturation, hemoglobin concentration, or an absorption coefficient inside an object and melanin concentration near the surface of an object) or designate a concept such as the “arrangement state of blood vessels in an object” and a “skin color.” As a measurement target, the doctor may designate not only a component constituting a living body but also a contrast agent (for example, ICG (indocyanine green)) or a medical agent applied to the living body, the pigment of a tattoo, a substance such as a perfume and cosmetics applied to the living body. The adjustment of the control parameters of the photoacoustic apparatus according to the imaging order is performed by an engineer or an information processing apparatus.

In the following description, “object information” is information showing the state of an object that has an influence on the acquisition of specific information by photoacoustic measurement. Particularly, the object information is information associated with the absorption of light by an object. The object information is mainly acquired from an electronic medical record but may be acquired by other methods.

A substance (for example, a contrast agent) administered or applied to a living body can not only be a target for the photoacoustic measurement as described above but also be an obstacle to the measurement of a substance constituting the living body (for example, the oxygen saturation of blood). In the latter case, the specific information is oxygen saturation, and the object information is contrast agent concentration.

In addition, melanin can not only be a target for the photoacoustic measurement but also be an obstacle to the photoacoustic measurement. That is, when a skin color is measured for a skin cancer examination or beauty diagnosis, the concentration or distribution of melanin is specific information. On the other hand, when the oxygen saturation or the like of a deep part of an object is measured, the concentration or distribution of melanin is object information that has an influence on the measurement of the oxygen saturation. In addition, the object information includes a change in the amount of the melanin of a skin due to sunburn or the like. The change in the amount of the melanin results in a change in the contrast between the melanin and a measurement target in an image. For this reason, control conditions are required to be changed.

The object information also includes the amount or state of body hairs, the states of beauty spots, flaws, or burns, the position or type of a medical device arranged inside a body, or the like. Further, the object information also includes information associated with previous photoacoustic measurement, for example, a past photoacoustic signal or photoacoustic image, light irradiating conditions or acoustic wave receiving conditions in past measurement, image reconfiguration conditions, or the like. Further, the object information also includes blood vessel brightness, melanin brightness, or the like read from a past photoacoustic image.

In the following description, “control conditions” indicate conditions associated with light irradiation or acoustic wave reception and are reflected on the control parameters of a light irradiating unit, a probe, or the like. An information processing apparatus may present the control parameters to an engineer and cause the engineer to set the control parameters in the apparatus, or may transmit a control signal to respective blocks to set the control parameters. As for the light irradiation, the control conditions include a wavelength, an irradiation light band, a light density, a light amount, the pulse width or interval of pulse light, an irradiation timing, the diffusion or convergence degree of light, or the like. As for the acoustic wave reception, the control conditions include the gain of an amplifier, a sampling interval, conditions associated with correction processing such as addition, a coefficient for determining image intensity, or the like. In addition, when a scanning mechanism for a probe or a light irradiating unit is provided, the control conditions also include the movement path or movement speed of the probe, a position at which acoustic waves are received, a position onto which light is irradiated, or the like.

Schematic Configuration of Apparatus

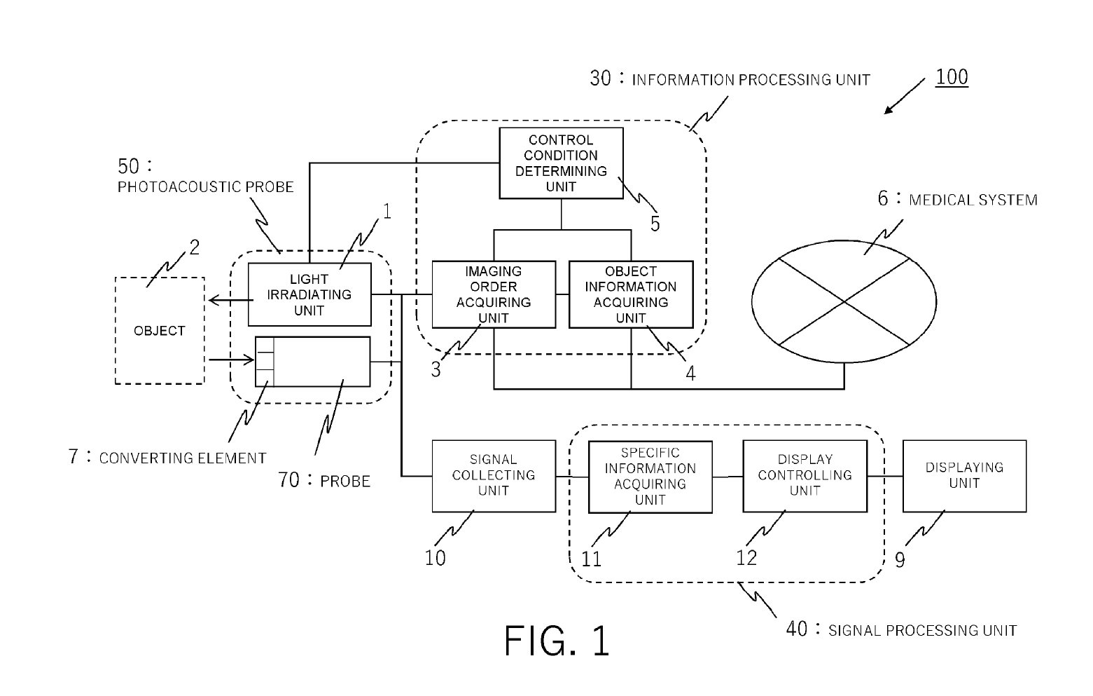

FIG. 1 shows the schematic configuration of a photoacoustic apparatus 100. The photoacoustic apparatus 100 includes an information processing unit 30, a photoacoustic probe 50 containing a light irradiating unit 1 and a probe 70, a signal collecting unit 10, a signal processing unit 40, and a displaying unit 9. The probe 70 contains a converting element 7. The information processing unit 30 contains an imaging order acquiring unit 3, an object information acquiring unit 4, and a control condition determining unit 5. The signal processing unit 40 contains a specific information acquiring unit 11 and a display controlling unit 12. A medical system 6 is a system capable of communicating with the photoacoustic apparatus 100 and contains at least any of an ordering system and an electronic medical record system.

The present invention is grasped as an invention associated with the photoacoustic apparatus 100. In this case, the photoacoustic apparatus 100 contains the information processing unit 30, the photoacoustic probe 50, and the signal processing unit 40. The photoacoustic apparatus 100 may also contain the displaying unit 9. The present invention is also grasped as an invention associated with the information processing apparatus 30 for determining control conditions. Hereinafter, preferred configuration examples of the respective constituents will be described.

Object

A photoacoustic apparatus primarily aims at making a diagnosis of a cancer, a blood vessel/skin disease, or the like in a human or an animal, keeping an eye on the developments of medical treatment, observing the state of a skin, or the like. Accordingly, a part of a living body, specifically a segment (a breast, an internal organ, a circulatory organ, a bone, a muscle, fat, a skin, or the like) of a human or an animal is assumed as an object. However, the object is not limited to this, and any substance can be the object so long as it absorbs light and emits acoustic waves.

In order to stabilize the shape of an object, it is preferable to hold the object with a holding member that is transmissive with respect to light and acoustic waves. A holding method includes a method for compressing the object with a plate or a method for holding a suspending breast with a cup-shaped holding member. As the material of the holding member, acryl or polymethyl pentene is preferable. In addition, a matching material for enhancing an acoustic matching effect is preferably arranged between the holding member and a probe and between the object and the holding member when the holding member is used. When the holding member is not used, the matching material is preferably arranged between the object and the probe. As the matching material, water, castor oil, ultrasonic gel, or the like is used.

Configuration about Light Irradiation

The light irradiating unit 1 of the photoacoustic probe 50 irradiates light from a light source (not shown) onto an object 2 via a light propagation member (not shown). Note that the light irradiating unit 1 and the probe 70 contained in the photoacoustic probe 50 may be physically integrated with each other or separated from each other. Examples of a physically-integrated example include a hand-held type probe that includes a light irradiating unit and a converting element, a probe in which a converting element is arranged on the inner peripheral surface of a hemispherical supporting body and a light irradiating unit is arranged at the pole part of a hemisphere, or the like.

The light source is preferably one capable of emitting the pulse light of about nanoseconds to micro seconds. Particularly, a pulse width of at least one nanosecond and not more than 100 nanoseconds is suitable. The light has preferably a wavelength of at least 400 nm and not more than 1600 nm. In imaging a deep part of a living body, a wavelength of at least 700 nm and not more than 1100 at which the light is less absorbed in the background tissues of the living body is preferable. On the other hand, in imaging blood vessels near the surface of the living body at high resolution, a visible light region is preferable. In addition, substance concentration such as oxygen saturation may be acquired by the use of a wavelength variable laser or by the combination of a plurality of light sources having different wavelengths.

The light source is preferably a laser that produces a high output, and various lasers such as a solid laser, a gas laser, a pigment laser, and a semiconductor laser may be used. In addition, a light-emitting diode or a flash lamp may be used instead of the laser. As the light propagating member, an optical member such as an optical fiber, a lens, a mirror, a prism, and a diffusion plate may be used. In guiding pulse light, the spot shape or light density of the pulse light may be changed by the optical member.

Configuration Associated with Acoustic-Wave Reception

The probe 70 of the photoacoustic probe includes the converting element 7. The converting element 7 receives photoacoustic waves generated inside or at the surface of an object and converts the received photoacoustic waves into an electrical signal. As the converting element 7, a piezoelectric element, a converting element using the resonance of light, a capacitance type converting element such as a CMUT, or the like may be used. In addition, the probe 70 may include a plurality of converting elements 7. In this case, the arrangement of the respective elements in a linear shape or two-dimensional shape enhances the precision of image reconfiguration. Moreover, the converting elements 7 may be three-dimensionally arranged by a hemispherical probe. The probe 70 corresponds to an acoustic wave receiving unit according to the present invention.

In addition, the photoacoustic apparatus may include a scanning mechanism that controls the relative position of the probe 70 with respect to an object. Alternatively, a hand-held type probe including a part gripped by a user may be used. In this case, the light irradiating unit 1 may be incorporated in the probe 70. Further, in the case of a photoacoustic microscope, the probe 70 of a focus type is preferably configured to mechanically move along the surface of an object. Further, the irradiation position of irradiation light and the probe 70 preferably move in synchronization with each other.

Information Processing Unit

The information processing unit 30 is a function block that performs processing associated with the determination of the control conditions of the photoacoustic apparatus 100. The information processing unit 30 may be constituted by a single information processing apparatus, or may be configured by the cooperation between a plurality of apparatuses. In addition, the information processing unit 30 and the signal processing unit 40 that will be described later may be mounted on the same information processing apparatus. As the information processing unit 30, a PC or a workstation that includes a CPU, a GPU, and a memory (a storage medium such as a ROM, a RAM, and a hard disk) and performs information processing according to an instruction by a program may be preferably used.

The imaging order acquiring unit 3 communicates with the medical system 6 to acquire information. From the ordering system of the medical system 6, an imaging order from a doctor may be acquired. In addition, the electronic medical record of an object may be acquired from an electronic medical record system. The imaging order contains a measurement target desired by the doctor.

The object information acquiring unit 4 acquires object information showing the state of an object that has an influence on the imaging of an ordered measurement target. In some cases, an imaging order contains the object information. In addition, the object information may be acquired using a device attached to the photoacoustic apparatus. Such a device includes a camera that optically images the surface of an object and an apparatus that acquires the absorption/scattering information of light near the surface of a living body by means of a near-infrared time decomposition spectral method. In addition, an operator such as a doctor and an engineer may be caused to input the object information using an inputting unit such as a mouse and a keyboard.

Alternatively, some object information is obtained based on the result of photoacoustic measurement. For example, melanin concentration at the surface of an object acquired by the photoacoustic measurement may be used to determine parameters for acquiring an image of blood vessels at a deep part of the object.

Based on the state of an object shown by object information, the control condition determining unit 5 determines control conditions so as to comply with an imaging order from a doctor. The control condition determining unit 5 may present preferable parameters to an engineer. In addition, the control condition determining unit 5 may transmit determined parameters to the light irradiating unit 1 or the probe 70. Moreover, when the control conditions are associated with light irradiation, acoustic wave reception, and a scanning timing, the control condition determining unit 5 transmits a control signal to respective blocks during photoacoustic measurement to control a timing.

Signal Collection Unit

The signal collecting unit 10 collects a time-series analog electrical signal output from each of the plurality of converting elements 7 for each channel and performs signal processing such as amplification, digital conversion, the storage of a digital signal, and correction. The signal collecting unit 10 is constituted by processing circuits such as an amplifier and an AD convertor. As the signal collecting unit 10, a circuit generally called DAS (Data Acquisition System) may be used. In addition, the signal collecting unit 10 may be arranged inside the probe 70. A signal output from the signal collecting unit 10 is sequentially stored in a memory.

Signal Processing Unit

The signal processing unit 40 is a function block that performs signal processing using an electrical signal output from the converting elements 7. The signal processing unit 30 may be constituted by a single information processing apparatus, or may be configured by the cooperation between a plurality of function blocks. As the signal processing unit 40, a PC or a workstation that includes a CPU, a GPU, a memory (a storage medium such as a ROM, a RAM, and a hard disk) and performs information processing according to an instruction by a program may be preferably used.

The specific information acquiring unit 11 reads an electrical signal output from the signal collecting unit 10 from a memory and performs image reconfiguration processing to generate specific information inside an object. For image reconfiguration, any known method such as universal back projection (UBP), filtered back projection (FBP), and a phase addition method (delay and sum) may be used.

For example, when the acquisition of an oxygen saturation distribution is requested in an imaging order, the light irradiating unit 1 irradiates the light of a plurality of wavelengths onto an object at different timings. The specific information acquiring unit 11 generates an initial sound pressure distribution derived from each of the wavelengths by the image reconfiguration. Then, the specific information acquiring unit 11 calculates an absorption coefficient distribution corresponding to the respective wavelengths using a light amount distribution estimated from an irradiation light amount and the absorption and scattering characteristics of the object. After that, the specific information acquiring unit 11 acquires an oxygen saturation distribution using a difference in the absorption spectrum between oxygenated hemoglobin and reduced hemoglobin.

Note that the photoacoustic microscope of a light focus type or an acoustic focus type is allowed to generate a specific information distribution without performing the image reconfiguration processing. Specifically, the photoacoustic microscope receives photoacoustic waves while causing the probe 70 and a light irradiation spot to relatively move with respect to the object 2. The photoacoustic microscope performs the envelope detection of an obtained electrical signal with respect to a temporal change and converts a time-axis direction into a depth direction in a signal for each light pulse to be plotted on space coordinates. Further, the photoacoustic microscope performs the processing for each scanning position.

The display controlling unit 12 generates image data caused to be displayed on the displaying unit 9 based on a specific information distribution generated by the specific information acquiring unit 11. At this time, the display controlling unit 12 may perform image processing such as brightness conversion, distortion correction, and logarithmic compression processing and the generation of an image item to be displayed together with the specific information distribution.

Displaying Unit

As the displaying unit 9, any displaying apparatus such as a liquid crystal display and an organic EL display may be used. The displaying unit 9 may be provided together with the photoacoustic apparatus 100, or may be provided separately from the photoacoustic apparatus 100.

Detailed Configurations

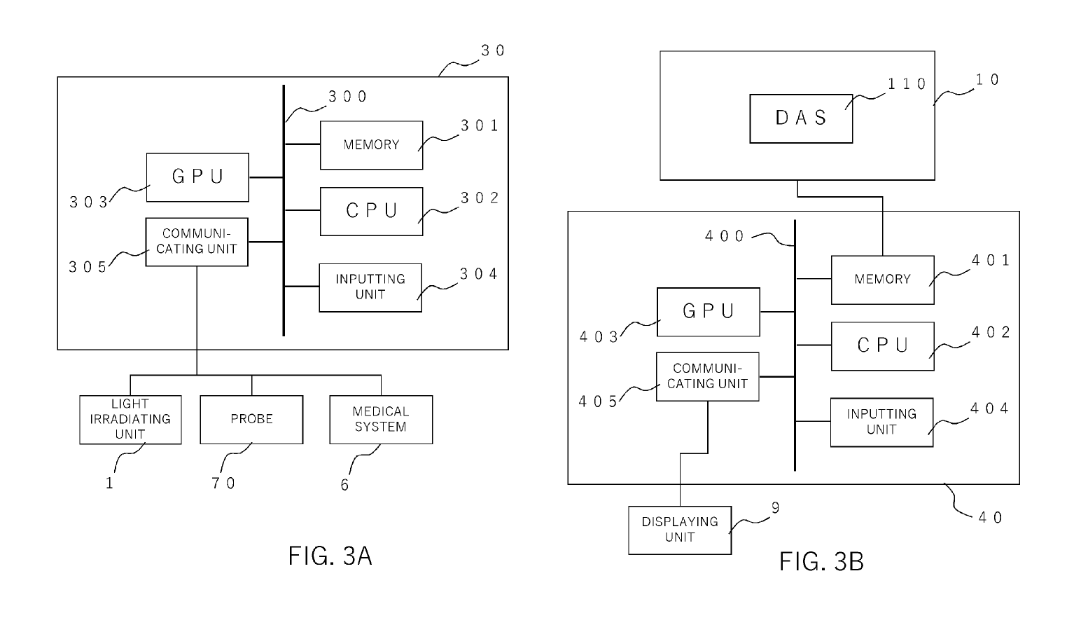

FIG. 3A is a schematic diagram showing an example of the structure of the information processing unit 30 and a relationship with the external apparatuses. The information processing unit 30 includes a memory 301, a CPU 302, a GPU 303, an inputting unit 304, and a communicating unit 305.

The CPU 302 bears a part of the functions of the imaging order acquiring unit 3, the object information acquiring unit 4, and the control condition determining unit 5. Specifically, the CPU 302 controls the respective configuration blocks of the information processing unit 30 via a system bus 300. In addition, the CPU 302 communicates with the ordering system, the electronic medical record system, or the like of the medical system 6 via the communicating unit 305 to acquire an imaging order or object information and stores the acquired information in the memory 301. Moreover, the CPU 302 performs calculation using an imaging order or object information stored in the memory 301. Further, the CPU 302 communicates with the light irradiating unit 1 or the probe 70 via the communicating unit 305 to set control parameters based on determined control conditions or control a timing for irradiating light or receiving acoustic waves.

The GPU 303 performs calculation in cooperation with the CPU 302 and particularly makes it possible to perform advanced calculation associated with image processing. As the memory 301, a ROM, a RAM, a hard disk, or the like is used. The inputting unit 304 is a user interface such as a mouse and a keyboard and used to input information by a doctor or an engineer.

FIG. 3B is a schematic diagram showing an example of the structure of the signal processing unit 40 and a relationship with the external apparatuses. The signal processing unit 40 includes a memory 401, a CPU 402, a GPU 403, an input unit 404, and a communicating unit 405. The signal collecting unit 10 includes a DAS 110. The DAS 110 bears a part of the functions of the signal collecting unit 40 and transfers a signal having been subjected to amplification or digital conversion to the memory 401.

In this example, the CPU 402 bears a part of the functions of the specific information acquiring unit 11 and the display controlling unit 12. Specifically, the CPU 402 controls the respective configuration blocks of the signal processing unit 40 via a system bus 400, generates image data showing a specific information distribution, and outputs the generated image data to the displaying unit 9. Particularly, the GPU 403 performs various image processing such as brightness conversion, distortion correction, and the extraction of a noticeable region on specific information. The functions of the other blocks are the same as those shown in FIG. 3A. Note that the information processing unit 30 and the signal processing unit 40 may be realized by the same information processing apparatus as described above.

Processing Flow

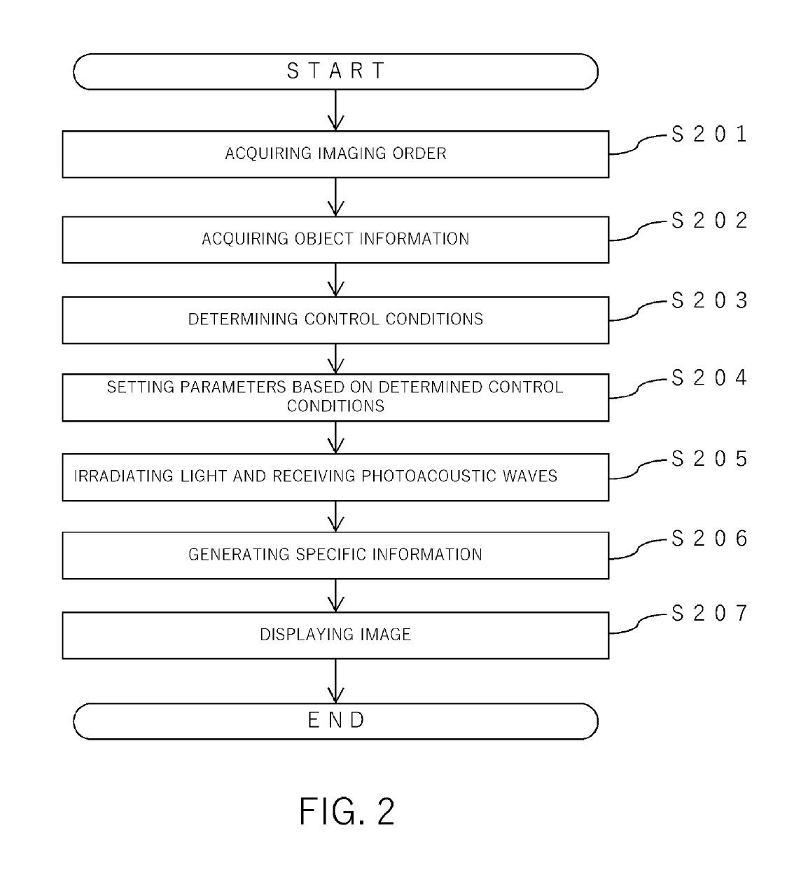

Next, the processing flow of the present embodiment will be described with reference to FIG. 2. The flow starts from a state in which an imaging order from the ordering system of the medical system 6 has been added to the imaging order acquiring unit 3 of the information processing unit 30 after the completion of preprocessing such as the start-up of the photoacoustic apparatus and the retention of an object.

In step S201, the imaging order acquiring unit 3 acquires the imaging order having been added to the ordering system. Here, it is assumed that the imaging order is the “acquisition of an oxygen saturation distribution inside the object.”

In step S202, the object information acquiring unit 4 acquires the state of the object that has an influence on the execution of the ordered imaging. Here, the object information acquiring unit 4 communicates with an electronic medical record system to acquire information associated with the reactivity of the object with respect to light. As the information associated with the reactivity with respect to the light, it is described in an electronic medical record that the object has photosensitivity.

In step S203, the control condition determining unit 5 determines light irradiating conditions and acoustic wave receiving conditions that comply with the imaging order based on the object information. For example, the control condition determining unit 5 sets an amount of light to be irradiated onto the object at a value smaller than a value established as a maximum allowable exposure amount in view of the photosensitivity of the object. Alternatively, when the imaging order contains information showing execution of light irradiation at a predetermined light amount and for a predetermined number of times, the control condition determining unit 5 causes the photoacoustic probe to perform the light irradiation at a light amount smaller than the ordered predetermined light amount and for the number of times greater than the ordered predetermined number of times in view of the object information showing the fact that the object has the photosensitivity.

In step S204, the control condition determining unit 5 sets the control parameters of the light irradiating unit 1 based on the determined conditions. In addition, since the intensity of photoacoustic waves reduces with a reduction in the light amount, the control condition determining unit 5 may increase the number of times of the light irradiation to improve an SN ratio or control the parameters of the signal collecting unit 10 to increase a gain.

In step S205, the light irradiating unit 1 irradiates light having a light amount smaller than an ordinary amount according to the set control parameters. The converting elements 7 of the probe 70 receive photoacoustic waves, and the signal collecting unit stores a digital signal in a memory. In step S206, the specific information acquiring unit 11 generates a specific information distribution. In step S207, the display controlling unit 12 generates display data and displays the generated display data on the displaying unit 9.

According to the flow, control conditions complying with an imaging order may be easily determined based on object information acquired from an electronic medical record or the like. Therefore, it becomes possible to present an image useful for making a diagnosis by a doctor. For the reference of imaging at a later date, the determined conditions may be newly stored in the electronic medical record or the like.

### Second Embodiment

In the present embodiment, the determination of control conditions when photoacoustic measurement in which an imaging order and object information are different from those of the above embodiment is performed will be described. The descriptions of the same configurations and the processing as those of the above embodiment will be simplified.

The content of the imaging order of the present embodiment is to make a melanin component inside an object into an image as a measurement target. The object information acquiring unit 4 acquires information associated with a background component inside the object as a substance that has an influence on specific information. Note that the background component indicates a component other than melanin that is a measurement target. Here, there is a likelihood that an amount of light reaching the melanin reduces when the background component absorbs a part of irradiation light, and that photoacoustic waves derived from the melanin are not transmitted due to photoacoustic waves generated from the background component. In view of this problem, the control condition determining unit 5 determines, based on the photographing order and the object information, control conditions so that the contrast between the melanin and the living-body background component is increased to obtain an image in which the melanin is emphasized.

An example of setting conditions by the control condition determining unit 5 will be described. First, there is a method in which a wavelength at which an absorption degree is made greatly different between the melanin and a background part is selected as the wavelength of irradiation light to increase a contrast in an image. In this case, the object information acquiring unit 4 acquires the absorption spectrum information of a main background component as object information. Specifically, the object information acquiring unit 4 acquires the type of the object such as a breast and a hand from an electronic medical record or an imaging order. Then, the object information acquiring unit 4 acquires the component ratio or the absorption spectrum information of the background component according to the type of the object. The use of the wavelength selected by the method increases the intensity of photoacoustic waves derived from the melanin and provides an image in which the melanin is emphasized.

Information associated with the component ratio or the light absorption spectrum of the background component may be acquired from an electronic medical record system or the Internet, may be contained in the imaging order, or may be stored in a memory in advance. A data format is arbitrarily, and a table showing an absorption ratio for each wavelength according to a measurement target or a background component may be, for example, used. The table may be discrete or continuous with respect to a wavelength. In the case of a discrete table, the object information acquiring unit 4 acquires an absorption ratio with respect to a specific wavelength by interpolation processing.

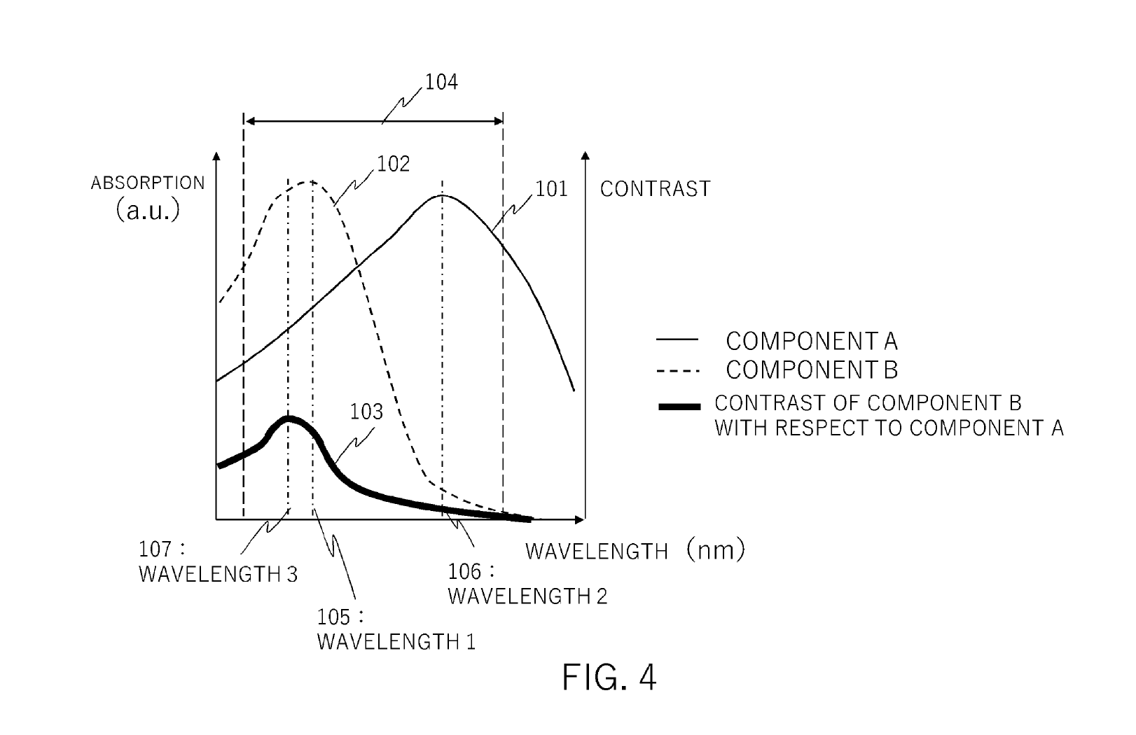

With reference to FIG. 4, a method for determining a wavelength at which a contrast in an image becomes maximum from absorption information with respect to respective wavelengths will be described. For simplification, only two components (a component A that is a background component and a component B that is a measurement target) are used. The horizontal axis and the vertical axis of the graph of FIG. 4 are a wavelength and an absorption degree, respectively. In the graph, an absorption spectrum 101 with respect to the respective wavelengths of the component A, an absorption spectrum 102 with respect to the respective wavelengths of the component B, and a contrast 103 of the component B with respect to the component A are shown. A reference numeral 104 shows the range of the wavelength of laser light capable of being irradiated by the photoacoustic apparatus. The absorption of the component A becomes maximum at a wavelength 2 (reference numeral 106), and the absorption of the component B becomes maximum at a wavelength 1 (reference numeral 105). The contrast 103 is calculated in such a manner that a contrast is calculated from absorption information at the respective wavelengths. It appears from the graph that a wavelength 3 (reference numeral 107) is only required to be selected to maximize the contrast of the component B with respect to the component A.

Besides, various control methods can be assumed. For example, a method in which the width of irradiation light is changed according to a region to be observed and a method in which image intensity is adjusted between a plurality of images so that the contrasts of melanin and a background part become the same are available. In addition, a method in which signal intensity is adjusted between a plurality of signals, a method in which the pulse width of a laser is changed according to the diameter of a blood vessel to be observed, or the like is available.

The component of a measurement target may be emphasized in an image, and any method is available to perform the emphasis of the component. For example, a method in which the brightness of the measurement target is increased, a method in which the color of the measurement target is made different from a background component, a method in which the region of the measurement target in the image is increased, a method in which the signal intensity of the background component is decreased, a method in which the intensity of the measurement target and the rest is adjusted, or the like is available. Thus, a doctor is allowed to easily visually recognize the measurement target.

Control conditions determined by the control condition determining unit 5 may be stored in an electronic medical record, a memory inside the information processing unit, or the like to be used in subsequent imaging. For example, when an irradiation wavelength is determined, the information processing unit communicates with an electronic medical record and writes the value of the wavelength in the electronic medical record. Thus, an irradiation wavelength may be easily acquired when the same object is imaged at the next time.

According to the present embodiment, information associated with the light absorption of a background component such as fat is used as object information when an image of a measurement target designated by an imaging order is generated, whereby it becomes possible to select a preferable wavelength. As a result, an image having an excellent contrast may be generated.

### Third Embodiment

In the present embodiment as well, the determination of control conditions when photoacoustic measurement in which an imaging order and object information are different from those of the above embodiments is performed will be described. The descriptions of the same configurations and the processing as those of the above embodiments will be simplified.

The information processing unit of the present embodiment communicates with an ordering system and an electronic medical record to acquire object information based on the information of one component and selects a wavelength at which the contrast of the one component becomes maximum. Here, an object is a living body, and a measurement target is ICG. The light source of the present embodiment is configured to be capable of covering a wide-band wavelength with a plurality of semiconductor lasers.

The content of the imaging order of the present embodiment is to make the arrangement state of blood vessels inside the object into an image. In addition, the object information acquiring unit 4 acquires, from the electronic medical record, the information that a contrast agent (ICG) was administered to the inside of the blood vessels of the object for imaging by another modality on the day before photoacoustic measurement. Moreover, the object information acquiring unit 4 acquires the administered amount and the administered time of the ICG, the ejection speed of the ICG by the object, or the like. The object information acquiring unit 4 calculates ICG concentration inside the blood vessels of the object at the present moment based on information acquired from the electronic medical record.

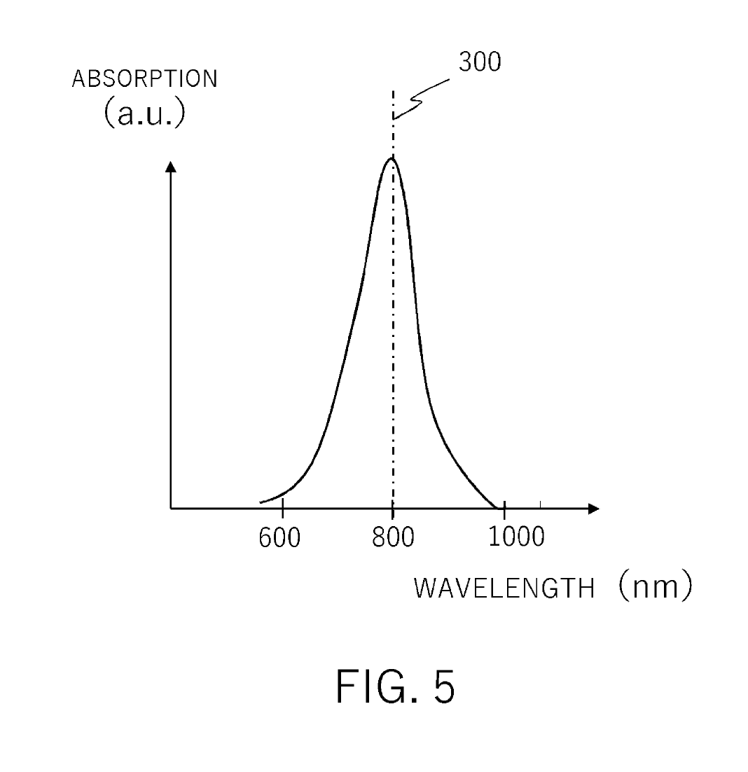

When the arrangement state of blood vessels is made into an image by a photoacoustic apparatus, an absorption coefficient, oxygen saturation, or the like is often used. However, if ICG exists inside a body, there is a likelihood that ordinary measurement assuming hemoglobin cannot be accurately performed. In view of this problem, the control condition determining unit 5 sets parameters for making the arrangement state of blood vessels into an image using the ICG. That is, the control condition determining unit 5 acquires the absorption spectrum of the ICG from a memory or the Internet. Then, the control condition determining unit 5 calculates a wavelength at which a contrast with respect to a background becomes maximum within the range of a wavelength at which a laser is allowed to be irradiated to determine the wavelength of irradiation light. FIG. 5 is a schematic diagram of the absorption spectrum of ICG. It appears that the absorption of the ICG becomes maximum near 800 nm shown by reference numeral 300, and that the contrast of the ICG becomes maximum.

As described above, a wavelength at which the contrast of ICG becomes maximum is selected based on the information of an imaging order and an electronic medical record in the present embodiment. Therefore, even if imaging with hemoglobin is difficult due to the influence of ICG remaining inside an object, the arrangement state of blood vessels may be accurately made into an image.

In the above example, the wavelength is determined using the absorption information of the one component (ICG). However, a wavelength may be determined using the absorption information of at least two components. For example, a wavelength at which the contrast of an ICG component with respect to melanin becomes maximum may be determined based on the absorption information of both ICG and the melanin inside a skin like the second embodiment.

### Modified Example

In the present embodiment, the wavelength at which the absorption degree of the ICG is high is selected. However, a wavelength at which absorption by ICG is small may be selected to suppress the fluorescence of the ICG. Thus, an image based on other substances (for example, hemoglobin) may be generated with a reduction in the influence of a contrast agent. As described in the present embodiment, it becomes possible to achieve both imaging with a contrast agent and imaging with a reduction in the influence of the contrast agent by the acquisition of the administration information of the contrast agent as object information.

### Fourth Embodiment

In the present embodiment as well, the determination of control conditions when photoacoustic measurement in which an imaging order and object information are different from those of the above embodiments is performed will be described. The descriptions of the same configurations and the processing as those of the above embodiments will be simplified.

In the imaging order of the present embodiment, the imaging of microscopic blood vessels inside an object is instructed. The object information acquiring unit 4 acquires the thickness (thinness) of blood vessels in a concerned region from an electronic medical record to be used as object information. The control condition determining unit 5 determines the pulse width of a laser based on the thickness of the blood vessels to be used as the control parameters of the light irradiating unit 1. For example, it is assumed that the default value of the pulse width of the laser is 30 ns. In addition, it is assumed that the thickness of the blood vessels is 50 μm smaller than a general value. In this case, the control condition determining unit 5 changes the pulse width of the laser to 15 ns to make relatively thin blood vessels into an image with fine resolution.

In the present embodiment, control conditions suitable for the imaging of microscopic blood vessels are set based on an imaging order and object information as described above. Therefore, an image with fine resolution is obtained even from microscopic blood vessels.

### Fifth Embodiment

In the present embodiment as well, the determination of control conditions when photoacoustic measurement in which an imaging order and object information are different from those of the above embodiments is performed will be described. The descriptions of the same configurations and the processing as those of the above embodiments will be simplified.

A measurement target ordered in the present embodiment is melanin at the surface of a living body. In the present embodiment, laser intensity or the gain of a signal is adjusted based on the absorption information of one component, whereby an image of which the image intensity with respect to a certain amount of an absorber does not change regardless of a change in a wavelength is generated. In addition, the image intensity may be adjusted.

Here, both eumelanin and pheomelanin coexist in a skin. Since the eumelanin and the pheomelanin have different absorption spectrums, the color of the skin changes according to the ratio between the eumelanin and the pheomelanin. In the present embodiment, melanin indicates a state in which both the eumelanin and the pheomelanin coexist, and the total amount of the melanin in which both the eumelanin and the pheomelanin are mixed together becomes specific information. In order to make the total amount of the melanin into an image, it is not preferable to use light having a wavelength at which the image intensity of one of the eumelanin and the pheomelanin becomes strong. In view of this problem, the object information acquiring unit of the present embodiment acquires the existence amounts of the eumelanin and the pheomelanin or the ratio between the eumelanin and the pheomelanin as object information.

In the present embodiment, it is assumed that a laser is a tunable laser. After the start of measurement, the photoacoustic apparatus sequentially irradiates the lasers of a first wavelength and a second wavelength onto an object and receives photoacoustic waves derived from each of light beams. Next, the specific information acquiring unit 11 performs calculation based on a difference in the absorption characteristics between the eumelanin and the pheomelanin to calculate an existence amount per unit area of each of the eumelanin and the pheomelanin. The calculated existence amounts are stored in a memory accessible from the information processing unit. Note that the existence amount distribution of each of the components at the surface of a living body may be calculated instead of the existence amounts per unit area. In this case, more accurate melanin distribution information may be acquired with a change in a wavelength for each place.

Subsequently, the control condition determining unit 5 refers to the calculated existence amounts of the eumelanin and the pheomelanin per unit area and selects a wavelength at which the absorption degrees of both the types of the melanin substantially match each other. Thus, melanin distribution information may be acquired without leaning to any of the types.

Note that irradiation light intensity at respective wavelengths when photoacoustic measurement is performed using a plurality of wavelengths instead of selecting one wavelength may be determined. Alternatively, the gain of the acoustic wave receiving unit may be adjusted for each wavelength based on the absorption information of the melanin to make the appearance of the melanin uniform. In addition, it is preferable to adjust image intensity.

According to the present embodiment, control conditions are controlled using object information calculated based on absorption spectrum information. Therefore, accurate melanin information may be acquired.

### Sixth Embodiment

In the present embodiment as well, the determination of control conditions when photoacoustic measurement in which an imaging order and object information are different from those of the above embodiments is performed will be described. The descriptions of the same configurations and the processing as those of the above embodiments will be simplified.

Measurement targets ordered in the present embodiment are the melanin amount of a skin and a total hemoglobin amount inside an object. Here, it is known that the melanin amount increases when the skin is exposed to ultraviolet rays or the like. Therefore, when photoacoustic measurement is performed for a plurality of times under the same control conditions to observe a temporal change, there is a likelihood that a change in the melanin amount has an influence on the measurement value of hemoglobin. In view of this problem, communication with an ordering system and an electronic medical record is performed based on the information of the two components of the melanin amount and the total hemoglobin amount to acquire object information.

Specifically, after the acquisition of the melanin amount and the total hemoglobin amount in first photoacoustic measurement, second photoacoustic measurement is performed with the elapse of a predetermined time. In the second measurement, the same control conditions as those in the first measurement are used to observe an increase and decrease in the melanin amount. Thus, a change in the melanin amount may be observed. In the second measurement, the comparison of an image of the total hemoglobin amount or the intensity of the melanin amount with respect to the intensity of the total hemoglobin amount may be made. When the melanin amount in the second measurement is greater than the melanin amount in the first measurement, there is a likelihood that hemoglobin intensity reduces due to the influence of melanin. In view of this problem, the control condition determining unit 5 changes control conditions to perform the measurement again. Thus, the total hemoglobin amount may be accurately acquired. That is, in the present embodiment, the melanin amount is not only a measurement target but also object information.

Note that image processing in which the total hemoglobin amount reflects the actual condition may be performed when respective images acquired in the two measurement are presented to a doctor. In addition, with the assumption that the hemoglobin amount at the same position in the same blood vessel does not almost change, the image intensity of one image may be changed to make the hemoglobin amounts at a certain blood vessel position constant to make a comparison of the melanin amount.

According to the present embodiment, even if there is a likelihood that a melanin amount in a skin changes in a plurality of measurement on the same patient, the comparison of a melanin amount or a total hemoglobin amount may be made based on the information of the two components of melanin and hemoglobin.

### Other Embodiments

The present invention is realized also by performing the following processing. That is, a program or an imaging recipe for realizing at least one of the functions of the above respective embodiments is supplied to a system or an apparatus via a network or various storage media. At least one processor in the computer of the system or the apparatus reads a program or imaging conditions to perform processing. In addition, the present invention may also be realized by a circuit (for example, an FPGA or an ASIC) that realizes at least one function.

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2017-253591, filed on Dec. 28, 2017, which is hereby incorporated by reference herein in its entirety.

1. A photoacoustic apparatus comprising:
a photoacoustic probe configured to include a light irradiating unit that irradiates light onto an object and an acoustic wave receiving unit that receives an acoustic wave generated from the object by the irradiation of the light;
a specific information acquiring unit configured to acquire specific information of the object based on the acoustic wave;
an order acquiring unit configured to acquire an order relating to the acquisition of the specific information from an ordering system;
an object information acquiring unit configured to acquire object information relating to the object from an electronic medical record; and
a control condition determining unit configured to determine a control condition of photoacoustic probe based on the order and the object information.

2. The photoacoustic apparatus according to claim 1, wherein,
when the order contains information indicating execution of light irradiation at a predetermined light amount and for a predetermined number of times and the object information contains information indicating a fact that the object has photosensitivity, the control condition determining unit is configured to determine the control condition so that the photoacoustic probe is caused to perform the light irradiation at a light amount smaller than the predetermined light amount and for the number of times greater than the predetermined number of times.

3. The photoacoustic apparatus according to claim 1, wherein
the object information is information relating to absorption of the light, which has an influence on the acquisition of the specific information, by a component inside the object.

4. The photoacoustic apparatus according to claim 3, wherein
the object information is information relating to a substance constituting the object, information relating to a substance administered or applied to the object, or information indicating reactivity of the object to the light.

5. The photoacoustic apparatus according to claim 1, further comprising:
a display controlling unit configured to control a display of an image based on the specific information on a displaying unit, wherein
the control condition determining unit is configured to determine the control condition so that the specific information of a measurement target designated by the order is emphasized in the image.

6. The photoacoustic apparatus according to claim 1, wherein
the object information acquiring unit is configured to acquire the object information using a device that optically images a surface of the object or a device to which an operator inputs the object information.

7. The photoacoustic apparatus according to claim 1, wherein
the control condition determining unit is configured to store the control condition in the electronic medical record.

8. An information processing apparatus comprising:
a control condition determining unit configured to acquire, from an ordering system, an order relating to acquisition of specific information of an object generated based on a photoacoustic wave generated when light is irradiated onto the object, acquire object information relating to the object from an electronic medical record, and determine a control condition of a photoacoustic probe to acquire the photoacoustic wave based on the order and the object information.

9. The information processing apparatus according to claim 8, wherein,
when the order contains information indicating execution of light irradiation at a predetermined light amount and for a predetermined number of times and the object information contains information indicating a fact that the object has photosensitivity, the control condition determining unit is configured to determine the control condition so that the photoacoustic probe is caused to perform the light irradiation at a light amount smaller than the predetermined light amount and for the number of times greater than the predetermined number of times.

10. An information processing method comprising:
acquiring, from an ordering system, an order relating to acquisition of specific information of an object generated based on a photoacoustic wave generated when light is irradiated onto the object;
acquiring object information relating to the object from an electronic medical record; and
determining a control condition of a photoacoustic probe to acquire the photoacoustic wave based on the order and the object information.

11. The information processing method according to claim 10, wherein,
when the order contains information indicating execution of light irradiation at a predetermined light amount and for a predetermined number of times and the object information contains information indicating a fact that the object has photosensitivity, the control condition is determined in the determination of the control condition so that the photoacoustic probe is caused to perform the light irradiation at a light amount smaller than the predetermined light amount and for the number of times greater than the predetermined number of times.

12. A non-transitory computer readable storage medium storing a program that causes a computer to perform the information processing method according to claim 10.

13. A non-transitory computer readable storage medium storing a program that causes a computer to perform the information processing method according to claim 11.
